(19) United States
(12) Patent Application Publication
(10) Pub. No.: US 2007/0238762 A1
(43) Pub. Date: Oct. 11, 2007

(54) Use of Antagonists of Hepatic Sympathetic Nerve Activity

(75) Inventor: Wilfred Wayne Lautt, Winnipeg (CA)

Correspondence Address: MERCHANT & GOULD PC, P.O. BOX 2903, MINNEAPOLIS, MN, 55402-0903, US

(73) Assignee: DiaMedica Inc., Winnipeg (CA)

(21) Appl. No.: 10/570,547

(22) PCT Filed: Sep. 15, 2004
(86) PCT No.: PCT/CA04/01682

§ 371 Date: Feb. 14, 2007

Related U.S. Application Data
(60) Provisional application No. 60/502,626, filed on Sep. 15, 2003.

(51) Int. Cl. A61K 31/417; A61K 31/135; A61K 31/44

(52) U.S. Cl. 514/352; 514/401; 514/646

(57) ABSTRACT

The present invention provides pharmaceutical compositions comprising antagonists of hepatic sympathetic activity and methods for using said pharmaceutical compositions for treatment of hyperglycemia, hyperinsulinaemia, hyperlipidaemia, hypertriglyceridaemia, diabetes, insulin resistance, impaired glucose metabolism, conditions of impaired glucose tolerance, conditions of impaired fasting plasma glucose, obesity, diabetic retinopathy, diabetic nephropathy, glomerulosclerosis, diabetic neuropathy, syndrome X, renal failure, sexual dysfunction, chronic stress, and anxiety.

### FIELD OF INVENTION

The present invention relates to pharmaceutical compositions and uses thereof for the treatment and prevention of disorders caused by or related to abnormal hepatic sympathetic nerve activity.

### BACKGROUND

Following a meal, hepatic parasympathetic nerves provide a permissive signal to the liver that regulates the ability of insulin to stimulate the release of a hormone, HISS, from the liver. HISS selectively stimulates glucose uptake and storage as glycogen in skeletal muscle and accounts for over one-half of the whole body glucose disposal that has previously been assumed to be a direct effect of insulin. Hepatic sympathetic nerves block the parasympathetic signal thus preventing the release of HISS and resulting in a 50% reduction in the glucose disposal effect of insulin. This condition is referred to as HISS-dependent insulin resistance (HDIR).

HISS action can be clinically diagnosed by determining the response to insulin in the fasted state and following re-feeding. The difference in the glucose disposal effect of an injection of insulin determined in the fed and fasted state represents the HISS- dependent component of insulin action. The glucose disposal produced in the fasted state is independent of HISS whereas the approximately doubled effect of insulin following a meal is due to both the HISS-dependent and HISS-independent component of insulin action with the difference between the two states being defined as the HISS-dependent component of insulin action.

HISS-dependent and HISS-independent insulin action can be most readily quantitated using the rapid insulin sensitivity test (RIST) which is a transient euglycemic clamp in response to a bolus administration of insulin. Normally insulin injection stimulates removal of glucose from the blood into storage sites with a resultant decrease in blood glucose level occurring. The RIST method uses variable glucose infusion rates to maintain the blood glucose level constant. The amount of glucose required to be administered in order to maintain the glycemic baseline is the index of insulin sensitivity and is referred to as the RIST index. The RIST index produced by this procedure consists of a HISS-dependent component and a HISS-independent component that can be readily differentiated by testing in the control fed state and then repeating the test after blockade of HISS release by any of a number of means including surgical denervation of the liver, blockade of hepatic muscarinic receptors, blockade of hepatic nitric oxide production, or blockade of hepatic cyclooxygenase. Eliminating HISS action by any of these procedures results in a reduction of the RIST index, in the fed state, of approximately 55%. That is, the glucose disposal effect that has been previously attributed to the direct action of insulin on a variety of tissues is actually mediated to a large extent by a hepatic insulin sensitizing process that has previously been unrecognized. This area has recently been reviewed (Lautt, 1999; Lautt, 2003). Blockade of HISS release results in HDIR. If HDIR is produced physiologically in response to fasting, these interventions do not produce any further decrease in insulin action.

HDIR is a normal and essential response to fasting. Insulin release occurs even in the fasted state and performs a number of growth regulating functions. Insulin is released in a pulsatile manner throughout the day with only approximately 50% of insulin release being regulated by food ingestion (Beyer et al., 1990). In the fasting state, it would be disadvantageous for insulin to cause a massive shifting of glucose from blood to skeletal muscle glycogen stores. The glucose disposal action in response to an injection of insulin decreases progressively to insignificance by 24 hours of fasting. This decrease in response to insulin represents a physiologically adjusted decrease in the HISS-dependent component as demonstrated by the observation that the HISS-independent (post-atropine or post-hepatic denervation) component of insulin action is similar in fed and 24-hour fasted rats.

In the immediate postprandial state, approximately 55% of the total glucose disposal effect of a bolus administration of insulin over a wide physiological range (5-100 mU/kg) is accounted for by HISS. By 18 hours of fasting, Sprague Dawley rats show HISS-dependent insulin action that accounts for only 26% of total insulin action (Lautt et al., 2001). The proportion of insulin action accounted for by HISS action remaining after 18 hours of fasting in cats is 35% (Xie & Lautt, 1995) and 25% in dogs (Moore et al., 2002). HISS action in rabbits accounts for approximately 44% of insulin action although the time since feeding was not stated (Porszasz et al., 2002). Fasting induces a 45% reduction in insulin action in mice (Latour & Chan, 2002). Preliminary results indicate that 62% of insulin action in the fed state is accounted for by HISS action in humans. This physiological regulation of HDIR is an appropriate response to fasting and, as such HDIR is a useful physiological state.

While HDIR is a useful physiological state in the fasted condition, failure to release HISS and the resultant HDIR in the fed state is suggested to account for the major metabolic disturbance seen in type 2 diabetes and many other conditions of insulin resistance. According to this model, post-meal nutrient processing normally results in approximately 80% of the glucose absorbed from a meal being stored in the large skeletal muscle mass of the body. Although HISS is released from the liver, it selectively stimulates glucose uptake into glycogen stores in skeletal muscle. Lack of HISS action results in a greatly impaired glucose disposal effect of insulin thus resulting in postprandial hyperglycemia. Additional insulin is released in response to the elevated glucose thus accounting for postprandial hyperinsulinemia in the type 2 diabetic. Insulin stimulates glucose uptake into adipose tissue and into the limited stores of the liver. When the glycogen stores in the liver are saturated, the remaining glucose is converted to lipid thus accounting for postprandial hyperlipidemia in the type 2 diabetic. The biochemical effects of hyperglycemia including the generation of free radicals has been suggested to account for the major non-metabolic pathologies common to diabetics including endothelial cell dysfunction, deposition of atherosclerotic plaques, blindness, renal failure, nerve damage, stroke, and hind limb amputation (Brownlee, 2001). HDIR has been shown to occur in chronic liver disease, fetal alcohol exposed adults, obesity, sucrose fed rats, hypertension, pregnancy and trauma.

Previous studies have focused on using pharmaceuticals to reverse HDIR based on restoring or potentiating the parasympathetic nerve function. Until now the mechanism by which the parasympathetic function is progressively decreased with fasting or is acutely triggered was unknown.

### SUMMARY OF THE INVENTION

The present invention provides a pharmaceutical composition as an antagonist of hepatic sympathetic activity comprising an a adrenergic antagonist and a β adrenergic antagonist.

In an embodiment, the pharmaceutical composition comprises an antagonist of hepatic sympathetic activity and an acetylcholine esterase antagonist. The present invention also provides a pharmaceutical composition comprising an antagonist of hepatic sympathetic activity and a phosphodiestrase antagonist.

The present invention further provides a pharmaceutical composition comprising an antagonist of hepatic sympathetic activity and at least one other drug used in the treatment of diabetes.

In an embodiment of the present invention, the antagonist of hepatic sympathetic activity is selected from a group comprising: an a adrenergic antagonist, a β adrenergic antagonist, and a mixture thereof.

The present invention provides a method of increasing skeletal muscle glucose uptake in a mammalian patient comprising administering an antagonist of hepatic sympathetic nerve activity. The antagonist of hepatic sympathetic activity may be selected from the group consisting of an a adrenergic antagonist and a β adrenergic antagonist.

The present invention also provides a method of reducing insulin resistance in a mammalian patient comprising administering an antagonist of hepatic sympathetic nerve activity. The antagonist of hepatic sympathetic activity may be selected from the group consisting of an α adrenergic antagonist and a β adrenergic antagonist.

The present invention further provides a method for the prevention, delay of progression or treatment of a disorder selected from a group comprising: hyperglycemia, hyperinsulinaemia, hyperlipidaemia, hypertriglyceridaemia, diabetes, insulin resistance, impaired glucose metabolism, conditions of impaired glucose tolerance, conditions of impaired fasting plasma glucose, obesity, diabetic retinopathy, diabetic nephropathy, glomerulosclerosis, diabetic neuropathy, syndrome X, renal failure, sexual dysfunction, chronic stress, and anxiety in a mammalian patient, comprising administering an antagonist of hepatic sympathetic activity.

The present invention still further provides a method for the prevention, delay of progression or treatment of a mammalian patient suffering a disorder selected from a group comprising: hyperglycemia, hyperinsulinaemia, hyperlipidaemia, hypertriglyceridaemia, diabetes, insulin resistance, impaired glucose metabolism, conditions of impaired glucose tolerance, conditions of impaired fasting plasma glucose, obesity, diabetic retinopathy, diabetic nephropathy, glomerulosclerosis, diabetic neuropathy, syndrome X, renal failure, sexual dysfunction, chronic stress, and anxiety, comprising administering a pharmaceutical composition wherein the pharmaceutical composition comprises an antagonist of hepatic sympathetic activity and a phosphodiesterase antagonist, an acetylcholine esterase antagonist, or a drug used in the treatment of diabetes.

### BRIEF DESCRIPTION OF THE FIGURES

FIG. 1 is a bar graph showing insulin sensitivity in rats prior to and following experimentally induced hemorrhage.

FIG. 2 is a bar graph showing insulin sensitivity in rats in the fasted state, in the fed state, and following administration of phentolamine and propanolol.

FIG. 3 is a graph showing insulin sensitivity in rats in the fasted state and following administration of phentolamine.

FIG. 4 is a bar graph showing insulin sensitivity in rats following administration of phentolamine and phentolamine in combination with arecoline.

### DETAILED DESCRIPTION

The present inventors have discovered that hepatic sympathetic nerves are capable of blocking hepatic parasympathetic nerve function and consequently removing the parasympathetic signal that is required in order for insulin to cause the release of HISS, i.e. activation of the hepatic sympathetic nerves or elevated levels of circulating catecholamines can lead to HDIR. The present inventors have also discovered that antagonism of hepatic sympathetic nerve activity is capable of restoring the parasympathetic signal and ameliorating HDIR. The inventors have determined methods of treating HDIR resulting from hepatic sympathetic nerve blockade of parasympathetic nerve function.

In light of these discoveries, the present invention provides pharmaceutical compositions useful for the prevention, delay in progression, and treatment of insulin resistance, and more specifically HISS dependent insulin resistance. As used herein, HISS dependent insulin resistance” (“HDIR”), is defined as a reduction in the response to insulin secondary to a failure of HISS action on glucose disposal. HDIR can be clinically diagnosed by measuring the response to insulin in the fasted stated and following re-feeding wherein the absence of an increase in glucose disposal in the fed state as compared to the fasted state, is diagnostic of HDIR.

The present invention provides novel methods of treatment and pharmaceutical compositions which employ an antagonist of hepatic sympathetic activity. As used herein, “hepatic sympathetic nerve antagonist” includes any composition which reduces or inhibits hepatic sympathetic nerve activity or the consequences of such activity including by the blockade of catecholamine receptors. The methods and pharmaceutical compositions of the present invention may be used to treat mammalian patients including human patients.

Preferably, the hepatic sympathetic nerve antagonist will comprise compounds which antagonize either pre- or post synaptic adrenergic receptors (“adrenergic receptor antagonist”). As used herein, the term “α-adrenergic antagonist” includes any composition which has a high degree of selectivity for α-adrenergic receptors. The term “β-adrenergic antagonist”, as used herein, includes any composition which has a high degree of selectivity for β-adrenergic receptors.

In one embodiment of the present invention, at least one adrenergic receptor antagonist is administered to a patient suffering impaired skeletal muscle glucose uptake or insulin resistance. Adrenergic receptor antagonists which may be used to practice the invention include but are not limited to: α-adrenergic antagonist such as prazosin, terazosin, doxazosin, phenoxybenzamine, phentoalamine, rauwolscine, yohimine, tolazoline; β-adrenergic antagonists such as metoprololol, acebutolol, alprenololol, atenolol, betaxolol, celiprolol, esmolol, propanolol, cartelolol, penbutolol, pindolol, timolol, butoxamine; and agents with mixed specificity such as carvedilol and labetolol.

Any suitable adrenergic receptor antagonist may be employed. As used herein, any pharmaceutical compound or composition is considered “pharmaceutically acceptable” if: (a) at the dose and method of administration to the patient, it is not acutely toxic, and does not result in chronic toxicity disproportionate to the therapeutic benefit derived from treatment, and (b) the dose and method of administration to the patient reduces insulin resistance in the patient.

One or more adrenergic receptor antagonists may be co-administered. In a preferred embodiment of the invention, the patient is co-administered an α- adrenergic receptor antagonist and a β-adrenergic antagonist. A non-limiting example of a suitable combination is the co-administration of phentoalamine and propanolol.

The present invention also provides useful pharmaceutical compositions comprising at least one antagonist of hepatic sympathetic activity and another compound for prevention and treatment of insulin resistance. The pharmaceutical compositions of the present invention can also be used for the prevention and treatment of other conditions, disorders and diseases including: hyperglycemia, hyperinsulinaemia, hyperlipidaemia, hypertriglyceridaemia, diabetes, insulin resistance, impaired glucose metabolism, conditions of impaired glucose tolerance, conditions of impaired fasting plasma glucose, obesity, diabetic retinopathy, diabetic nephropathy, glomerulosclerosis, diabetic neuropathy, syndrome X, renal failure, sexual dysfunction, chronic stress, and anxiety.

While the invention is not limited to a particular model or mechanism of action, it appears that in normal individuals, the parasympathetic response to feeding results in the release of acetylcholine which activates muscarinic receptors in the liver. This activation leads to increased production of nitric oxide which stimulates guanyl cyclase activity, resulting in increased levels of cyclic guanosine monophosphate which acts in stimulating the release of HISS. Feeding also results in elevated hepatic glutathione levels which is essential for the parasympathetic signal to permit insulin to cause HISS release. Interruption of any component of this system can result in reduction or abolishment of the parasympathetic response to feeding. Accordingly, insulin resistance and related disorders may be the result of not only abnormal parasympathetic activity but also abnormal sympathetic activity. Thus, the invention provides pharmaceutical compositions and uses thereof for relieving insulin resistance and related disorders and diseases, which correct both hepatic sympathetic and parasympathetic function.

In some instances, parasympathetic function in response to feeding is impaired due to decreased acetylcholine production or release: In view of the inventor's recent discovery concerning hepatic sympathetic blockade of the parasympathetic feeding response, the present invention provides a novel pharmaceutical composition comprising an antagonist of hepatic sympathetic activity and an acetylcholine esterase antagonist. The inventors have previously described the use of acetylcholine esterase antagonists for the treatment of insulin resistance in U.S. patent application Ser. No. 10/350,478.

Any suitable combination of at least one antagonist of hepatic sympathetic activity and at least one acetylcholine esterase antagonists can be used. Preferably the antagonist of hepatic sympathetic nerve actions will be an adrenergic receptor antagonist and more preferably, be a combination of an α-adrenergic receptor antagonist and a β-adrenergic antagonist. Acetylcholine esterase antagonists which can be used to practice the invention include but are not limited to: donepezil, galathamine, rivastigme, tacrine, physostigme, neostigme, edrophonium, pyridostigme, demarcarium, phospholine, metrifonate, zanapezil, and ambenonium.

In some instances, parasympathetic function in response to feeding is impaired due to decreased levels of cGMP or decreased responsiveness to cGMP. In view of the inventor's recent discovery concerning hepatic sympathetic blockade of the parasympathetic feeding response, the present invention provides a novel pharmaceutical composition comprising a hepatic sympathetic activity antagonist and a phosphodiesterase antagonist. The inventors have previously described the use of phosphodiesterase antagonists for the treatment of insulin resistance in U.S. patent application Ser. No.10/350,478.

Any suitable combination of at least one antagonist of hepatic sympathetic activity and at least one phosphodiesterase antagonists can be used. Preferably the antagonist of hepatic sympathetic activity will be an adrenergic receptor antagonist and more preferably, be a combination of an α-adrenergic receptor antagonist and a β-adrenergic antagonist. Phosphodiesterase antagonists which can be used to practice the invention include but are not limited to: vinopocetine, zaprinast, dipyridamole, sildenafil, theophylline, aminophylline, isobutylmethyl xanthine anagrelide tadalafil, dyphylline, vardenafil, cilostazol, caffiene, milirone, amrinone pimobendan, cilostamide, enoximone, teroximone, vesmarinone, rolipharm, and R020-1724.

In some instances, it will be desirable to administer an antagonist of hepatic sympathetic activity with other drugs used in the treatment of diabetes, non-limiting examples of which are provided in Table 1. For example, due to a failure of feeding to elevate hepatic glutathione levels, in some instances the sympathetic antagonist should be used in combination with compounds to elevate hepatic glutathione.

TABLE 1
Drugs Used in the Treatment of Diabetes

| a. | Insulin and insulin analogues | |
|---|---|---|
| b. | Type II Diabetes Drugs | |
| | i. | Sulfonylurea agents |

| | |
|---|---|
| 1. | First Generation |

| | |
|---|---|
| a. | tolbutamide |
| b. | acetohexamide |
| c. | tolazamide |
| d. | chlorpropamide |

| | |
|---|---|
| 2. | Second Generation |

| | |
|---|---|
| a. | glyburide |
| b. | glipizide |
| c. | glimepiride |

| | |
|---|---|
| ii. | Biguanide agents |

| | |
|---|---|
| 1. | metformin |

| | |
|---|---|
| iii. | Alpha-glucosidase inhibitors |

| | |
|---|---|
| 1. | acarbose |
| 2. | miglitol |

| | |
|---|---|
| iv. | Thiazolidinedione agents (insulin sensitizers) |

| | |
|---|---|
| 1. | rosiglitazone |
| 2. | pioglitazone |
| 3. | troglitazone |

| | |
|---|---|
| v. | Meglitinide agents |

| | |
|---|---|
| 1. | repaglinide |

| c. | Cholinergic agonists | |
|---|---|---|
| | i. | acetylcholine |
| | ii. | methacholine |
| | iii. | bethanechol |
| | iv. | carbachol |
| | v. | pilocarpine hydrochloride |
| | vi. | arecoline |
| d. | Nitric oxide donors | |
| | i. | products or processes to increase NO synthesis in the liver |
| | | (increasing NO synthase activity) |
| | | Variety I |

| | |
|---|---|
| 1. | SIN-1 |
| 2. | Molsidamine |

Variety II - nitrosylated forms of:

| | |
|---|---|
| 1. | N-acetylcysteine |
| 2. | cysteine esters |
| 3. | L-2-oxothiazolidine-4-carboxolate (OTC) |
| 4. | gamma glutamylcystein and its ethyl ester |
| 5. | glutathione ethyl ester, glutathione isopropyl ester |
| 6. | lipoic acid |
| 7. | cysteine |
| 8. | cystine |
| 9. | methionine |
| 10. | S-adenosylmethionine |

| | ii. | Products or processes to reduce the rate of NO degradation |
|---|---|---|
| | | in the liver |
| | iii. | Products or processes to provide exogenous NO or an |
| | | exogenous carrier or precursor which is taken up and |
| | | releases NO in the liver, antioxidants |
| e. | Antioxidants | |
| | i. | vitamin E |
| | ii. | vitamin C |
| | iii. | 3-morpholinosyndnonimine |
| f. | Glutathione increasing compounds | |
| | i. | N-acetylcysteine |
| | ii. | cysteine esters |
| | iii. | L-2-oxothiazolidine-4-carboxolate (OTC) |
| | iv. | gamma glutamylcystein and its ethyl ester |
| | v. | glutathione ethyl ester, glutathione isopropyl ester |
| | vi. | lipoic acid |
| | vii. | cysteine |
| | viii. | cystine |
| | ix. | methionine |
| | x. | S-adenosylmethionine |

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

The pharmaceutical compositions of the present invention may also include various other components which provide additional therapeutic benefit, act to affect the therapeutic action of the pharmaceutical composition, or act towards preventing any potential side effects which may be posed as a result of administration of the pharmaceutical composition. Exemplary pharmaceutically acceptable components or adjuncts which are employed in relevant circumstances include antioxidants, free radical scavenging agents, peptides, growth factors, antibiotics, bacteriostatic agents, immunosuppressives, anticoagulants, buffering agents, anti-inflammatory agents, anti-pyretics, time release binders, anaesthetics, steroids, vitamins, and minerals.

The precise dose for any of the pharmaceutical compositions of the present invention will depend on a number of factors which will be apparent to those skilled in the art and in light of the disclosure herein. In particular these factors include: the identity of the compounds to be administered, the formulation, the route of administration employed, the patient's gender, age, and weight, and the severity of the condition being treated. Methods for determining dosage and toxicity are well known in the art with studies generally beginning in animals and then in humans if no significant animal toxicity is observed. The appropriateness of the dosage can be assessed by monitoring insulin resistance using the RIST protocol as set out in Lautt et al, 1998. Where the dose provided does not cause insulin resistance to decline to normal or tolerable levels, following at least three days of treatment, the dose can be increased. The patient should be monitored for signs of adverse drug reactions and toxicity, especially with regard to liver and cardiovascular function.

For oral administration of adrenergic receptor antagonists twice daily doses can be administered wherein each dose is preferably between 0.01 mg/kg body weight and 100 mg/kg body weight. When the adrenergic receptor antagonist is phentoalamine, each dose is preferably between 20-40 μg/kg/min. Where the adrenergic receptor antagonist used is propanolol, each dose is preferably 0.1-0.2 μg/kg/min.

Where one or more adrenergic receptor antagonists are co-administered or where an adrenergic receptor antagonist is administered with a phosphodiesterase antagonist, an acetylcholine esterase antagonist or a diabetes drug, reference should be made to toxicity studies performed according to standard techniques known in the art and relating to the compounds to be administered. The precise formulation of the pharmaceutical compositions of the present invention should be determined in view of toxicity studies conducted in accordance to standard techniques known in the art and relating to the compounds to be administered. Combinations of compounds known to interact adversely and which result in toxicity should not be used.

The pharmaceutical compositions of the present invention may be administered so as to maintain a relatively constant level of the pharmaceutical composition in the liver at all times. Alternatively, the pharmaceutical composition may be administered to have it peak when blood glucose is high, such as after a meal, so as to allow glucose uptake at that time. Where toxicity is a concern, it may be desirable to keep levels low until blood glucose levels become elevated above normal levels or to administer the drugs only before each meal.

The pharmaceutical compositions of the present invention can be targeted to the liver of the patient thereby eliminating deleterious systemic effects. The pharmaceutical compositions can be conjugated to bile salts or albumin for preferential delivery to the liver. Alternatively, the pharmaceutical compositions can be encapsulated within liposomes which are preferentially targeted to the liver. The pharmaceutical compositions of the present invention can be administered either in active form or as precursor which is metabolized by to the active form by enzymes in the liver. Where the pharmaceutical composition is targeted to the liver, the dosage may be reduced.

### EXAMPLE ONE

### Evidence of Sympathetic Suppression of Parasympathetic Dependent HISS Release

Acute hemorrhage results in the activation of hepatic sympathetic nerves and the release of adrenal catecholamines, which results in the redundant control of glycogenolysis in the liver. The control is referred to as redundant in that the hyperglycemia that occurs following glycogen breakdown and release of glucose into the bloodstream in the stress situation is produced normally as long as either the hepatic sympathetic nerves or the adrenal glands are functioning normally. However, if both systems are eliminated, no such hyperglycemic response occurs. Acute stress results in the suppression of insulin release although this is unexpected as high blood glucose levels are usually associated with an increased release of insulin. In the case of trauma, however, the elevated blood glucose levels provide a high quality fuel for the insulin-independent central nervous system. As such, it would be disadvantageous to simultaneously release hepatic glucose stores and insulin which would simply transfer the glucose back out of the blood for re-storage in tissues thus producing a futile cycle and not reserving the glucose for fuel supply to the brain.

In order to determine whether HISS played a role in controlling glycogenolysis in response to acute trauma, fully anesthetized, fed, male, Sprague Dawley rats were prepared surgically according to the standard animal preparation used to conduct a rapid insulin sensitivity test (RIST) (Lautt et al., 1998). A control RIST was conducted to demonstrate full insulin sensitivity. Blood was then withdrawn at a rate of 0.5 ml/min to decrease arterial pressure to 50 mm Hg. Further blood removal was done as required to maintain pressure at 50 mm Hg for 5 minutes whereupon no further blood was withdrawn. Once plasma glucose levels stabilized, a RIST was then repeated.

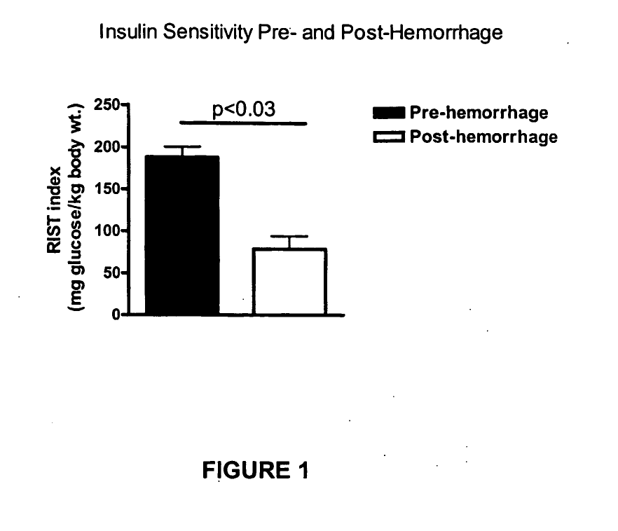

As shown in FIG. 1, insulin action in the fed state was suppressed by 56% following the hemorrhage indicating that acute trauma results in the blockade of HISS release and consequently, HDIR.

### EXAMPLE TWO

### Role of Sympathetic Nerves in the Progressive Decrease of HISS Release Following Liquid Test Meal and Subsequent Fasting

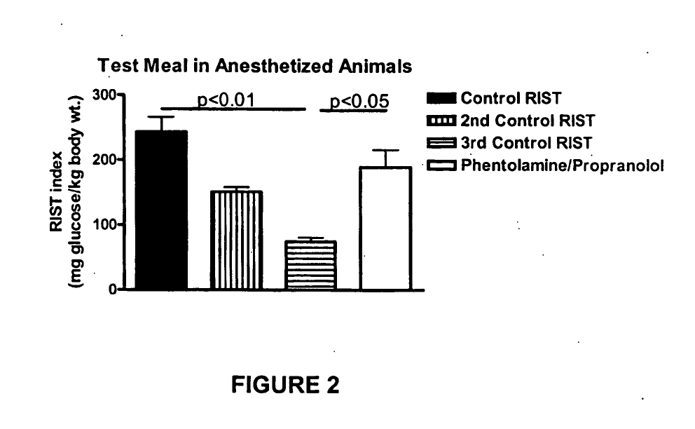

Conscious, male, Sprague Dawley rats were gavaged with 10 ml/kg of a mixed liquid test meal. The animals were anesthetized with pentobarbital sodium and a standard surgical preparation was performed as described in Example One. Insulin sensitivity was assessed immediately using the RIST methodology and resulted in a normal fed response as shown in FIG. 2.

A second RIST was then performed approximately 1 hour later and resulted in a significant decrease in the HISS-dependent component of insulin action.

A third RIST was conducted within 3 hours of the gavage. The results indicated that the feeding signal had been virtually eliminated by the time of the third RIST. This transient feeding signal provided a useful tool to determine the mechanism by which the parasympathetic signal was decreased.

Adrenergic receptor blockers for both alpha (phentolamine, 20-40 μg/kg/min) and beta receptors (propranolol 0.1-0.2 μg/kg/min) were then administered as a constant i.v. infusion and a fourth RIST was carried out and was shown to be significantly restored toward levels seen in the fed state. These results demonstrate that HDIR induced by fasting following a liquid test meal is reversed by adrenergic receptor blockade.

### EXAMPLE THREE

### Role of Sympathetic Nerves in the Progressive Decrease of HISS Release Following 24 Hour Fast

Male, Sprague Dawley rats were fed normal rat pellets and then fasted for a 24-hour period (with free access to water) prior to administration of pentobarbital sodium anesthesia and standard RIST methodology surgical preparation as described in Example One.

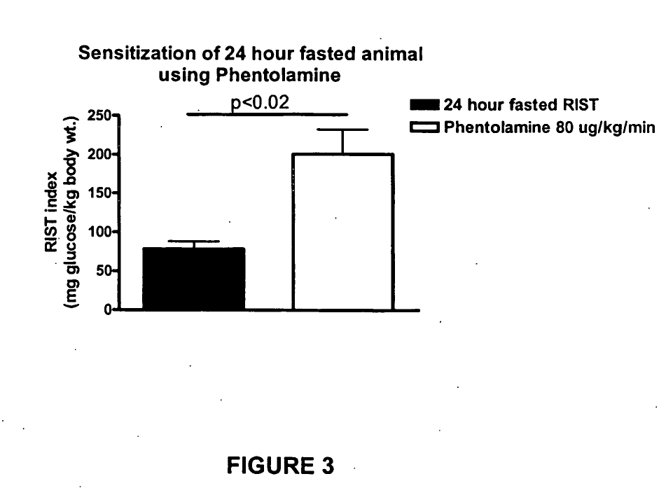

These animals showed typical HDIR induced by fasting. Insulin action was significantly restored toward normal levels by constant i.v. infusion of phentolamine as shown in FIG. 3. A tonic sympathetic tone is developed as the period of fasting progresses and results in a progressive suppression of the parasympathetic nerves thereby removing the permissive signal from the parasympathetic nerves that allows insulin to cause HISS release.

### EXAMPLE FOUR

### Role of Sympathetic Nerves in the Progressive Decrease of HISS Release Following 18 Hour Fast

Male, Sprague Dawley rats were fed normal rat pellets and then fasted for a 18-hour period (with free access to water) prior to administration of pentobarbital sodium anesthesia and standard RIST methodology surgical preparation as described in Example One.

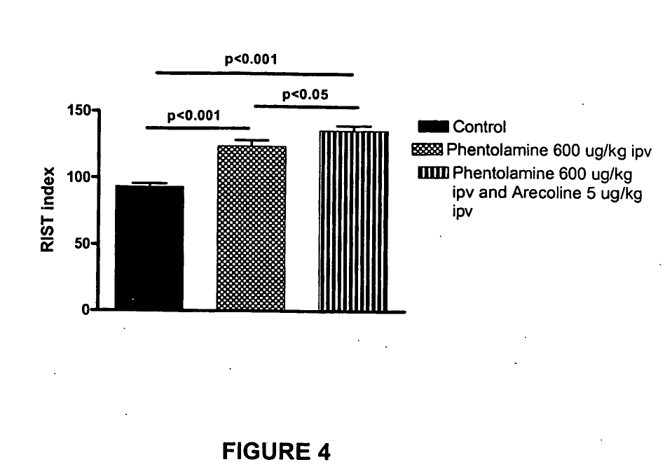

Following fasting, the animals were administered a bolus dose of 600 μg/kg ipv of phentolamine or a bolus dose of 600 μg/kg ipv of phentolamine and 5 μg/kg ipv of arecoline. As seen in FIG. 4, animals administered either phentolamine alone or phentolamine in combination with arecoline showed restoration of insulin action, with the combination therapy showing enhanced restoration of insulin action.

Although the present invention has been described with reference to illustrative embodiments, it is to be understood that the invention is not limited to these precise embodiments, and that various changes and modifications may be effected therein by one skilled in the art. All such changes and modifications are intention to be encompassed in the appended claims.

### REFERENCES

Beyer J, Krause U, Dobronz A, Fuchs B, Delver J R & Wagner R (1990). Assessment of insulin needs in insulin-dependent diabetics and healthy volunteers under fasting conditions. Horm Metab Res Suppl 24, 71-77.
Brownlee M (2001). Biochemistry and molecular cell biology of diabetic complications. Nature 414, 813-819.
Latour M G & Chan C C (2002). A rapid insulin sensitivity test (RIST) in the anesthetized mice (Abstract). Diabetes 51 (Suppl. 2), A422.
Lautt W W (1999). The HISS story overview: a novel hepatic neurohumoral regulation of peripheral insulin sensitivity in health and diabetes. Can J Physiol Pharmacol 77, 553-562.
Lautt W W (2003). New paradigm for insulin resistance: the HISS story. In: Atherosclerosis, Hypertension and Diabetes. Eds: G. N. Pierce, M. Nagano, P. Zahradka, and N. S. Dhalla. Kluwer Academic Publishers, Bost. Chapter 21, pp.263-276.
Lautt W W, Macedo M P, Sadri P, Takayama S, Ramos F D & Legare D J (2001). Hepatic parasympathetic nerve-dependent control of peripheral insulin sensitivity is determined by feeding and fasting: dynamic control of HISS-dependent insulin action. Am J Physiol 281, G29-G36.
Lautt, W. W., Wang, X., Sadri, P., Legare, D. J., and Macedo, M. P. (1998). Rapid insulin sensitivity test (RIST). Can. J. Physiol. Pharmacol. 76: 1080-1086. Moore M C, Satake S, Baranowski B, Hsieh P S, Neal D W & Cherrington A D (2002). Effect of hepatic denervation on peripheral insulin sensitivity in conscious dogs. Am J Physiol Endocrinol Metab 282, E286-E296.
Porszasz R, Legvari G, Nemeth J, Literati P, Szolcsanyi J & Szilvassy Z (2002). The sensory nitrergic nature of the hepatic insulin sensitizing substance mechanism in conscious rabbits. Eur J Pharmacol 443, 211-212.
Xie H & Lautt W W (1995). Induction of insulin resistance by cholinergic blockade with atropine in the cat. J Auton Pharmacol 15, 361-369.

1-30. (canceled)

31. A pharmaceutical composition comprising an antagonist of hepatic sympathetic activity selected from the group consisting of: an α-adrenergic antagonist, a β-adrenergic antagonist, and a mixture thereof.

32. The pharmaceutical composition according to claim 31, wherein the antagonist of hepatic sympathetic activity is a mixture of an α-adrenergic antagonist and a β adrenergic antagonist.

33. The pharmaceutical composition according claim 31, further comprising an acetylcholine esterase antagonist.

34. The pharmaceutical composition according to claim 33, wherein the acetylcholine esterase antagonist is selected from the group consisting of: donepezil, galathamine, rivastigme, tacrine, physostigme, neostigme, edrophonium, pyridostigme, demarcarium, phospholine, metrifonate, zanapezil, and ambenonium.

35. The pharmaceutical composition according to claim 31, further comprising a phosphodiestrase antagonist.

36. The pharmaceutical composition according to claim 35, wherein the phosphodiesterase antagonist is selected from the group comprising: vinopocetine, zaprinast, dipyridamole, sildenafil, theophylline, aminophylline, isobutylmethyl xanthine anagrelide tadalafil, dyphylline, vardenafil, cilostazol, caffiene, milirone, amrinone pimobendan, cilostamide, enoximone, teroximone, vesmarinone, rolipharm, and R020-1724.

37. The pharmaceutical composition according to claim 31, further comprising at least one other drug used in the treatment of diabetes.

38. The pharmaceutical composition according to claim 37, wherein the other diabetes drug is selected from a group comprising of: insulin, insulin analogues, sulfonylurea agents, tolbutamide, acetohexamide, tolazamide, chlorpropamide, glyburide, glipizide, glimepiride, biguanide agents, metformin, alpha-glucosidase inhibitors, acarbose, miglitol, thiazolidinedione agents (insulin sensitizers), rosiglitazone, pioglitazone, troglitazone, meglitinide agents, repaglinide, cholinergic agonists, acetylcholine, methacholine, bethanechol, carbachol, pilocarpine hydrochloride, arecoline, nitric oxide donors, products or processes to increase NO synthesis in the liver (increasing NO synthase activity), SIN-1, molsidamine, N-acetylcysteine, cysteine esters, L-2oxothiazolidine-4-carboxolate (OTC), gamma glutamylcystein and its ethyl ester, glutathione ethyl ester, glutathione isopropyl ester, lipoic acid, cysteine, cystine, methionine, S-adenosylmethionine, products or processes to reduce the rate of NO degradation in the liver, products or processes to provide exogenous NO or an exogenous carrier or precursor which is taken up and releases NO in the liver, antioxidants, vitamin E, vitamin C, 3-morpholinosyndnonimine, glutathione increasing compounds, N-acetylcysteine, cysteine esters, L-2-oxothiazolidine-4-carboxolate (OTC), gamma glutamylcystein and its ethyl ester, glutathione ethyl ester, glutathione isopropyl ester, lipoic acid, cysteine, cystine, methionine, and S-adenosylmethionine.

39. The pharmaceutical composition according to claim 31, wherein the antagonist of hepatic sympathetic activity is an α-adrenergic antagonist.

40. The pharmaceutical composition according to claim 31, wherein the antagonist of hepatic sympathetic activity is a β-adrenergic antagonist.

41. The pharmaceutical composition according to claim 31, wherein the antagonist of hepatic sympathetic nerve activity is selected from the group comprising: prazosin, terazosin, doxazosin, phenoxybenzamine, phentolamine, rauwolscine, yohimbine, tolazoline, metoprololol, acebutolol, alprenolol, atenolol, betaxolol, celiproplol, esmolol, propanolol, carteolol, penbutolol, pindolol, timolol, butoxamine, carvedilol, labetolol, and mixtures thereof.

42. The pharmaceutical composition of claim 31, further comprising a pharmaceutically acceptable liver-targeting substance.

43. The pharmaceutical composition of claim 42, wherein the pharmaceutically acceptable liver-targeting substance is selected from the group consisting of: bile salts, albumin, liposomes, and a mixture thereof.

44. A method of increasing skeletal muscle glucose uptake in a mammalian patient comprising administering an antagonist of hepatic sympathetic nerve activity.

45. The method according to claim 44, wherein the antagonist of hepatic sympathetic activity is selected from the group consisting of: an α adrenergic antagonist, a β adrenergic antagonist, and a mixture thereof.

46. The method according to claim 44, wherein the antagonist of hepatic sympathetic activity is an α-adrenergic antagonist.

47. The method according to claim 44, wherein the antagonist of hepatic sympathetic activity is a β-adrenergic antagonist.

48. The method according to claim 44, wherein the antagonist of hepatic sympathetic activity comprises an α-adrenergic antagonist and a β-adrenergic antagonist.

49. The method according to claim 44, wherein the antagonist of hepatic sympathetic nerve activity is selected from the group consisting of: prazosin, terazosin, doxazosin, phenoxybenzamine, phentolamine, rauwolscine, yohimbine, tolazoline, metoprololol, acebutolol, alprenolol, atenolol, betaxolol, celiproplol, esmolol, propanolol, carteolol, penbutolol, pindolol, timolol, butoxamine, carvedilol, labetolol, and mixtures thereof.

50. The method according to claim 44, wherein the antagonist of hepatic sympathetic activity is targeted to the liver using albumin.

51. The method according to claim 44, wherein the antagonist of hepatic sympathetic activity is targeted to the liver using a plurality of liposomes.

52. The method according to claim 44, wherein the antagonist of hepatic sympathetic activity is targeted to the liver using bile salts.

53. The method according to claim 44, wherein the mammalian patient is a human.

54. A method of reducing insulin resistance in a mammalian patient comprising administering an antagonist of hepatic sympathetic nerve activity.

55. The method according to claim 54, wherein the insulin resistance is hepatic insulin-sensitizing substance (HISS) dependent.

56. A method according to claim 54, wherein the antagonist of hepatic sympathetic activity is selected from the group consisting of: an α-adrenergic antagonist, a β-adrenergic antagonist, and a mixture thereof.

57. The method according to claim 54, wherein the antagonist of hepatic sympathetic activity is an α-adrenergic antagonist.

58. The method according to claim 54, wherein the antagonist of hepatic sympathetic activity is a β-adrenergic antagonist.

59. The method according to claim 54, wherein the antagonist of hepatic sympathetic activity comprises an α-adrenergic antagonist and a β-adrenergic antagonist.

60. The method according to claim 54, wherein the antagonist of hepatic sympathetic nerve activity is selected from the group consisting of: prazosin, terazosin, doxazosin, phenoxybenzamine, phentolamine, rauwolscine, yohimbine, tolazoline, metoprololol, acebutolol, alprenolol, atenolol, betaxolol, celiproplol, esmolol, propanolol, carteolol, penbutolol, pindolol, timolol, butoxamine, carvedilol, labetolol, and mixtures thereof.

61. The method according to claim 54, wherein the antagonist of hepatic sympathetic activity is targeted to the liver using albumin.

62. The method according to claim 54, wherein the antagonist of hepatic sympathetic activity is targeted to the liver using a plurality of liposomes.

63. The method according to claim 54, wherein the antagonist of hepatic sympathetic activity is targeted to the liver using bile salts.

64. The method according to claim 54, wherein the mammalian patient is a human.

65. A method for delay of progression or treatment of: hyperglycemia, hyperinsulinaemia, hyperlipidaemia, hypertriglyceridaemia, diabetes, insulin resistance, impaired glucose metabolism, conditions of impaired glucose tolerance, conditions of impaired fasting plasma glucose, obesity, diabetic retinopathy, diabetic nephropathy, glomerulosclerosis, diabetic neuropathy, syndrome X, renal failure, sexual dysfunction, chronic stress, or anxiety in a mammalian patient, comprising administering an antagonist of hepatic sympathetic activity.

66. The method according to claim 65, wherein the antagonist of hepatic sympathetic activity is selected from the group consisting of: an α adrenergic antagonist, a β adrenergic antagonist, and a mixture thereof.

67. The method according to claim 65, wherein the antagonist of hepatic sympathetic activity is an α-adrenergic antagonist.

68. The method according to claim 65, wherein the antagonist of hepatic sympathetic activity is a β-adrenergic antagonist.

69. The method according to claim 65, wherein the antagonist of hepatic sympathetic activity comprises an α adrenergic antagonist and a β-adrenergic antagonist.

70. The method according to claim 65, wherein the antagonist of hepatic sympathetic nerve activity is selected from the group consisting of: prazosin, terazosin, doxazosin, phenoxybenzamine, phentolamine, rauwolscine, yohimbine, tolazoline, metoprololol, acebutolol, alprenolol, atenolol, betaxolol, celiproplol, esmolol, propanolol, carteolol, penbutolol, pindolol, timolol, butoxamine, carvedilol, labetolol, and mixtures thereof.

71. The method according to claim 65, wherein the antagonist of hepatic sympathetic activity is targeted to the liver using albumin.

72. The method according to claim 65, wherein the antagonist of hepatic sympathetic activity is targeted to the liver using a plurality of liposomes.

73. The method according to claim 65, wherein the antagonist of hepatic sympathetic activity is targeted to the liver using bile salts.

74. The method according to claim 65, wherein the mammalian patient is a human.

75. A method for delay of progression or treatment of a mammalian patient suffering from: hyperglycemia, hyperinsulinaemia, hyperlipidaemia, hypertriglyceridaemia, diabetes, insulin resistance, impaired glucose metabolism, conditions of impaired glucose tolerance, conditions of impaired fasting plasma glucose, obesity, diabetic retinopathy, diabetic nephropathy, glomerulosclerosis, diabetic neuropathy, syndrome X, renal failure, sexual dysfunction, chronic stress, or anxiety, comprising administering the pharmaceutical composition of claim 31.

76. A method according to claim 75, wherein the pharmaceutical composition is targeted to the liver using albumin.

77. The method according to claim 75, wherein the pharmaceutical composition is targeted to the liver using a plurality of liposomes.

78. The method according to claim 75, wherein the pharmaceutical composition is targeted to the liver using bile salts.

79. The method according to claim 75, wherein the mammalian patient is human.
